(19) United States
(12) Patent Application Publication
(10) Pub. No.: US 2024/0079131 A1
(43) Pub. Date: Mar. 7, 2024

(54) SYSTEM AND METHOD FOR REMOTELY CONTROLLING MINIATURE DEVICE WITHIN A PATIENT

(71) Applicant: BIONAUT LABS LTD., Herzliya (IL)

(72) Inventors: Kishore Kumar KONDABATNI, Simi Valley, CA (US); Michael KARDOSH, Kiryat Ono (IL); SueHyun CHO, Torrance, CA (US); Florent CROS, Los Angeles, CA (US); Michael SHPIGELMACHER, Los Angeles, CA (US); Alex KISELYOV, San Diego, CA (US)

(73) Assignee: BIONAUT LABS LTD., Herzliya (IL)

(21) Appl. No.: 18/240,599

(22) Filed: Aug. 31, 2023

Related U.S. Application Data
(60) Provisional application No. 63/403,166, filed on Sep. 1, 2022.

(51) Int. Cl. G16H 40/67; G16H 20/30

(52) U.S. Cl. CPC G16H 40/67; G16H 20/30

(57) ABSTRACT

A system for facilitating performing a therapeutic activity at a predetermined treatment site in a patient is provided. The system comprises an elongated miniature device extending along a longitudinal axis spanning between a distal cutting end formed with a sharp cutting portion, and a blunt proximal abutting end. The elongated body comprises a substantially non-convex bottom surface extending substantially between the cutting end and the abutting end. The system further comprises a driving device configured to generate a varying magnetic field to remotely control motion of the miniature device.

### CROSS-REFERENCE TO RELATED APPLICATIONS

This application claims the benefit of U.S. Ser. No. 63/403,166, filed on Sep. 1, 2022, which is hereby incorporated by reference in its entirety.

### TECHNOLOGICAL FIELD

The presently disclosed subject matter relates to systems and miniature device configured to navigate within a patient to perform a therapeutic activity at a predetermined treatment site therewithin, and in particular to such systems which use magnetic fields to direct operation of miniature devices within a patient.

### BACKGROUND

Remote control of medical devices moving inside the human body can be useful for a variety of purposes, including delivery of therapeutic payloads, diagnostics or surgical procedures. Such devices may include microscale or nanoscale robots, medical tools, “smart pills,” etc. Such devices may be able to move in the body either through self-propulsion or an external propulsion mechanism. Accurate location and tracking of such devices may be necessary to ensure their proper functioning at the right anatomical location, and more specifically accurate delivery of the therapeutic payloads and/or diagnostics substances.

### SUMMARY

According to an aspect of the presently disclosed subject matter there is provided a system for facilitating performing a therapeutic activity at a predetermined treatment site in a patient, the system comprising:
an elongated miniature device extending along a longitudinal axis spanning between a distal cutting end formed with a sharp cutting portion, and a blunt proximal abutting end, the elongated body comprising a substantially non-convex bottom surface extending substantially between the cutting end and the abutting end; and
a driving device configured to generate a varying magnetic field to remotely control motion of the miniature device.

The miniature device may define first and second lateral planes being perpendicular to each other and mutually perpendicular to the longitudinal axis, wherein the widest width of the miniature device along the direction of the first lateral plane is at least twice the largest height of the miniature device along the direction of the second lateral plane.

For each point along a majority of the longitudinal axis, the width of the miniature device along the direction of the first lateral plane may be at least twice the height of the miniature device along the direction of the second lateral plane.

The bottom surface of the miniature device may be concave.

The bottom surface of the miniature device may be substantially planar.

In a direction perpendicular to the longitudinal axis, the width of the bottom surface may span a majority of the width of the miniature device, along a majority of the length of the miniature device.

The width of the bottom surface may be the width of the miniature device, along a majority of the length of the miniature device.

The cutting portion of the miniature device may be covered by a coating configured to dissolve within the patient after a predetermined amount of time has passed, thereby exposing the cutting portion.

The cutting portion of the miniature device may comprise one or more barbs configured to inhibit removal of cutting portion from a tissue pierced thereby.

The miniature device may comprise a shoulder formed proximally to and facing the cutting portion.

The miniature device may comprise a distal cutting element comprising the cutting end, a proximal abutting element comprising the abutting end, and a linking element connecting the cutting and abutting elements, the linking element being configured to selectively disconnect the cutting and abutting elements.

The linking element may comprise a material configured to dissolve within the patient after a predetermined amount of time has passed, thereby disconnecting the cutting and abutting elements.

The miniature device may comprise an external thread formed at least on a distal end thereof.

The miniature device may comprise a hydrogel configured, when exposed to water, to extend the miniature device along the longitudinal axis.

The miniature device may be covered with a lubricious coating.

The miniature device may comprise a magnetic material.

The magnetic material may be magnetized along the longitudinal axis.

The driving device may be designed to generate the varying magnetic to move the miniature device to a predefined location in the patient, and to rotate it at the location.

The driving device may be designed to generate the varying magnetic to rotate the miniature device such that the cutting portion thereof contacts a treatment site of a tissue in the patient, and the abutting end bears upon a contra location of a tissue opposite the treatment site, thereby facilitating piercing at the treatment site by the cutting portion.

The tissue opposite the treatment site may be a hard tissue, such as bone.

The driving device may be designed to generate the varying magnetic to move the miniature device within the patient such that it is oriented substantially along its longitudinal axis, wherein the abutting end of the miniature device constitutes a leading edge thereof, at least while moving toward the treatment site.

The therapeutic activity may comprise delivery of one or more therapeutic components.

The therapeutic activity may comprise performing one or more cutting operations.

The therapeutic activity may comprise treatment of a glioblastoma multiform.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 schematically illustrates a system according to the presently disclosed subject matter for remotely maneuvering a miniature device thereof within a patient;

FIG. 2 is a perspective view of an example of a miniature device of the system illustrated in FIG. 1;

FIGS. 3 through 7A & 7B illustrate different examples of a miniature device of the system illustrated in FIG. 1; and

FIGS. 8A and 8B illustrate an example of a use of the miniature device of the system illustrated in FIG. 1.

### DETAILED DESCRIPTION

In the following detailed description, numerous specific details are set forth in order to provide a thorough understanding of the presently disclosed subject matter. However, it will be understood by those skilled in the art that the presently disclosed subject matter may be practiced without these specific details. In other instances, well-known methods, procedures, and components have not been described in detail so as not to obscure the presently disclosed subject matter.

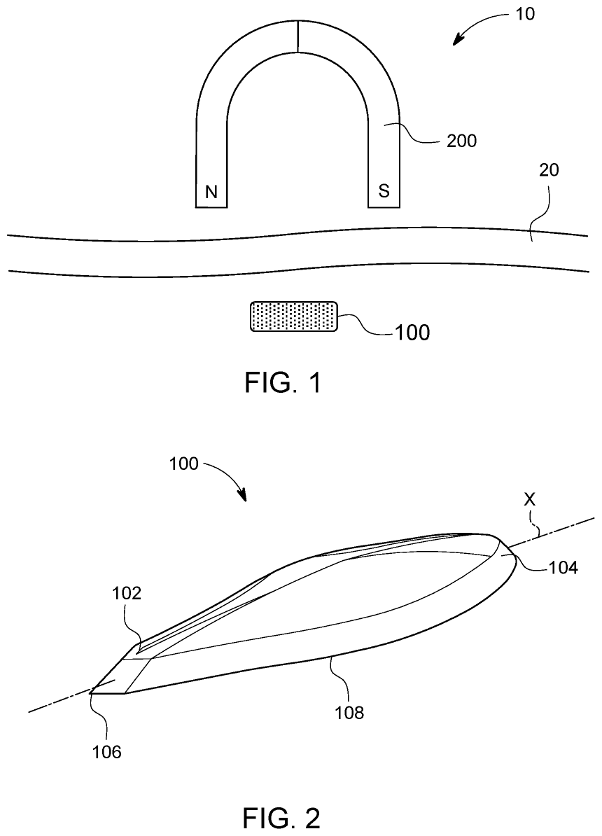

As illustrated in FIG. 1, there is provided a system 10 configured to facilitate performing a therapeutic activity at a predetermined location within a patient's body (hereinafter “treatment site”). According to some examples, the therapeutic activity comprises delivery of one or more chemical compounds of medicinal, diagnostic, evaluative, and/or therapeutic relevance, one or more small molecules, biologics, cells, one or more radioisotopes, one or more vaccines, etc. (hereinafter “therapeutic component”), for example via body fluids, an anatomic lumen, and/or soft tissue to the treatment site.

According to other examples, the therapeutic activity comprises performing a cutting operation at the treatment site.

It will be appreciated that for the sake of the present description, the term “treatment site” may be used herein the present description and appended claims to refer to the location where the system 10 acts, even if the location which is treated is located elsewhere. For example, the system 10 may be configured to perform a cutting operation at a location in order to access an adjacent location to deliver a therapeutic component thereto. In such a case, the location which undergoes the cutting operation may be referred to herein as the “treatment site,” even though it is not the ultimate recipient of the therapeutic component. In some cases, both the location at which the cutting operation is performed, as well as the location which is the ultimate recipient of the therapeutic component, may each be described as a “treatment site,” for example interchangeably within a single description of a therapeutic activity; in such cases, one having ordinary skill in the art will appreciate from context which location is referred to by each use of the term “treatment site.”

According to some examples, the system 10 is configured to facilitate delivery of a therapeutic component along a path adjacent one or more meninges of the brain. According to some examples, at least a portion of the path is in the subarachnoid space (SAS) between the arachnoid mater and the pia mater. According to other examples, at least a portion of the path is in the subdural space (SDS) between the dura mater and the arachnoid mater.

The system 10 comprises a miniature device 100 and a driving device, schematically indicated at 200, the patient being indicated schematically at 20. The miniature device 100 is configured to carry the therapeutic component. The driving device 200 is configured to be operated to configured to generate a varying magnetic field to remotely, i.e., from a location exterior to a patient's body, control the motion of the miniature device within the patient, for example as is known in the art.

According to some embodiments, characteristics of the magnetic field generated by the driving device 200, for example including, but not limited to, distance, directionality, intensity, gradient, time dependence/independence, etc., may be controlled by a user in order to remotely control the motion of the miniature device 100.

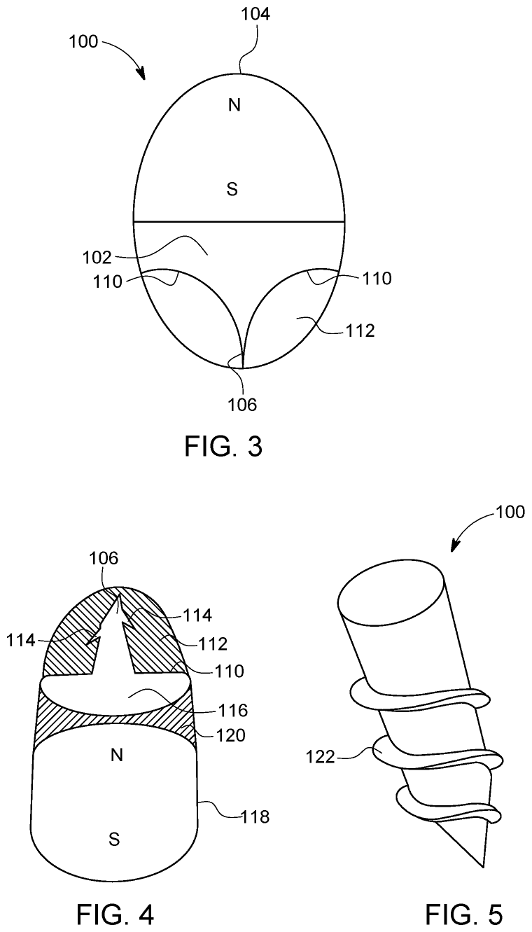

According to some examples, for example as illustrated in FIG. 2, the miniature device 100 is formed as an elongated body having a distal cutting end 102 and a proximal abutting end 104. A longitudinal axis X is defined spanning the length of the miniature device 100 between the distal cutting end 102 and the proximal abutting end 104. The miniature device 100 may be covered with a lubricious coating, for example on a portion of or on all of its surface, thereby facilitating movement thereof within the patient.

The miniature device 100 comprises a magnetic material, for example a permanent magnet and/or an electromagnet, configured to be acted on by the magnetic field generated by the driving device 200 to drive it. According to some examples, the magnetic material of the miniature device 100 is magnetized along the longitudinal axis X, i.e., with the north and south poles being disposed at opposite ends of the miniature device 100 along the longitudinal axis.

It will be appreciated that the while the miniature device 100 is described herein as being formed as a “body,” this is not to be construed as limiting either the present disclosure, specific examples using the term “body,” or claims reciting the term “body,” to a monolithic or unitary element. Rather, according to some examples the miniature device 100 may comprise two or more elements connected and/or configured to operate together, permanently and/or temporarily, without departing from the scope of the presently disclosed subject matter, mutatis mutandis.

The cutting end 102 is configured for performing a cutting operation (including, but not limited to, cutting, puncturing, lacerating, fenestrating, etc.) at the treatment site in the patient. Accordingly, it comprises a sharp cutting portion 106, for example facing in the distal direction. The cutting portion 106 is a suitable size and/or sharpness of the therapeutic activity for which it is designed, e.g., including, inter alia, movement to the treatment site.

According to some examples, the cutting portion 106 is surrounded by a coating configured to dissolve within the patient after a predetermined amount of time has passed, thereby exposing the cutting portion. Accordingly, risk of damage to tissue by the cutting portion 106 as the miniature device 100 travels to the treatment site is minimized.

The miniature device 100 may carry or be configured to carry a therapeutic component. According to some examples, the miniature device 100 comprises a cavity (not illustrated) for carrying therewithin the therapeutic component. The hollow may extend through the cutting portion 106 for delivery of the therapeutic component therethrough. According to some examples, the miniature device 100 comprises an arrangement, for example as is known in the art, configured to deliver the therapeutic component, e.g., selectively, at the treatment site.

As will be described below, the abutting end 104 is configured for abutting against a surface and/or tissue in the patient disposed opposite the treatment site. Accordingly, it is blunt, i.e., formed without features which are configured to perform a cutting operation or are otherwise sharp. It will be appreciated that the determination of what constitutes “sharp” may be dependent on the intended use for which the miniature device 100 is designed, for example based on the characteristics of the surface and/or tissue against which it is designed to abut, the amount of force with which it is designed to so abut, etc.

Typically, the bluntness of the abutting end 104 may be determined based on the sharpness of the cutting portion 106, e.g., one having skill in the art recognizing that not only is the abutting end blunter than the cutting portion, but that it qualitatively and/or categorically blunt with respect to the sharpness of the cutting portion.

A bottom surface 108 of the miniature device 100, spanning between the cutting end 102 and the abutting end 104, may be substantially non-convex. According to some examples, the bottom surface 108 is substantially planar. According to some examples, the bottom surface 108 may be concave, for example in a direction along the longitudinal axis X and/or in a direction perpendicular thereto and parallel to the bottom surface (i.e., along the width of the miniature device 100). The degree of the concavity of the bottom surface 108 may be chosen so as to provide an increased (e.g., compared to a planar surface) conformity of the shape of the bottom surface to a portion of the brain over which the miniature device 100 is designed to traverse as it moves within the patient.

It will be appreciated that the bottom surface 108 may be considered “substantially non-convex” even if it has some degree of convexity, e.g., bulging slightly, if the amount of convexity is much less (i.e., the radius of curvature is much more) than that of a circle perpendicular to the longitudinal axis X and circumscribing the miniature device 100.

The bottom surface 108 may constitute a significant portion of the width of the miniature device 100. According to some examples, its width is a majority of the width of the miniature device 100 along a majority of its length. Typically, the bottom surface 108 is the widest part of the miniature device 100 along a majority of its length. According to some examples, the bottom surface 108 is the widest part of the miniature device 100 along its entire length.

According to some examples, the miniature device 100 may have a substantially flattened shape, i.e., its width is at least twice its height (wherein the width is taken in a direction perpendicular to the longitudinal axis X and parallel to the bottom surface 108, and the height is taken in a direction mutually perpendicular to the longitudinal axis X and bottom surface 108). According to other examples, the width of the miniature device 100 is at least three times, at least four times, or at least five times its height.

The dimensions of the miniature device 100 may be provided based on the path within the patient along which it is designed to move and/or the location within the patient at which it is designed to act.

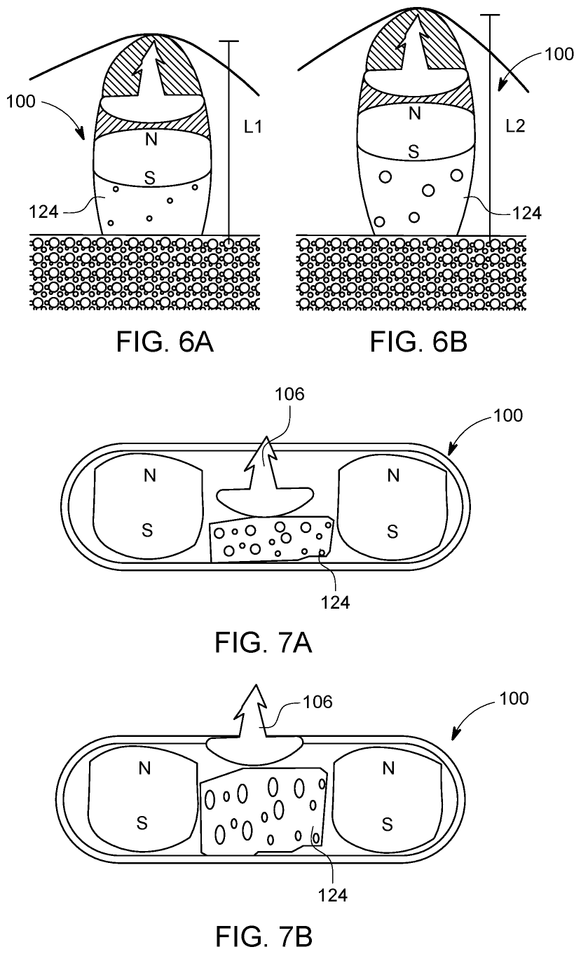

According to some examples, for example as illustrated in FIG. 3, the miniature device 100 is formed with a shoulder 110 formed proximally to the cutting portion 106 and facing it. The shoulder 110 may serve to limit penetration of the miniature device 100 when performing a cutting operation.

According to some examples, the cutting portion 106 may be covered by a coating configured to dissolve within the patient after a predetermined amount of time has passed, thereby exposing the cutting portion. This may increase patient safety while the miniature device 100 moves within the patient to the treatment site, e.g., by preventing inadvertent cutting by the cutting portion 106.

For example, as further illustrated in FIG. 3, the space surrounding the cutting portion 106 may be filled in with a filler material 112, constituting the coating, and being configured to dissolve within the patient, thereby facilitating positioning of the miniature device 100, for example as described below with reference to an illustrated in FIGS. 7A and 7B, before exposure of the cutting portion. This may increase patient safety while the miniature device is positioned for its cutting operation by preventing inadvertent cutting. In addition, the filler material 112 may be used to impart a shape to the miniature device 100 which is more suitable for rotation and/or movement within the patient.

According to some examples, such as illustrated in FIG. 4, the cutting device 106 may comprise one or more barbs 114 configured to inhibit removal thereof from a tissue it has pierced.

As further illustrated in FIG. 4, according to some examples the miniature device 100 may comprise a cutting element 116 comprising the cutting end 102, an abutting element 118 comprising the abutting end 104, and a linking element 120 connecting the cutting and abutting elements 116, 118. The miniature device 100 according to these examples may optionally be formed with shoulders 110 and/or the cutting portion 106 thereof may optionally be covered by a coating such as the filler material 112, such as described above with reference to and illustrated in FIG. 3. According to these examples, the linking element 120 may be configured to selectively disconnect the cutting and abutting elements 116, 118. According to some examples, the linking element 120 is made of a material which is configured to dissolve within the patient after a predetermined amount of time has passed, thereby disconnecting the cutting and abutting elements 116, 118. This may be useful to facilitate removal of part of the miniature device 100 while part of it remains at the treatment site within the patient.

It will be appreciated that while FIG. 4 illustrates an example of a cutting device 106 with barbs 114 as well as of a miniature device comprising cutting and abutting elements 116, 118 connected by a linking element, this is for convenience of disclosure only. In practice, the miniature device 100 may be provided in accordance with any one or both of the examples described above with reference to and illustrated in FIG. 4 without departing from the scope of the presently disclosed subject matter, mutatis mutandis.

According to some examples, such as illustrated in FIG. 5, the miniature device 100 may be formed with an external thread 122 formed at its distal end. The external thread 122 may constitute and/or cooperate with the cutting element 106 to perform a cutting operation. According to some examples, such as illustrated in FIGS. 6A and 6B, the miniature device 100 may be configured to extend along its longitudinal axis X. For example, it may comprise a hydrogel element 124 configured to expand when exposed to water (including water-like liquids and/or liquids containing water, such as cerebrospinal fluid within the SAS). When the hydrogel element 124 expands, the overall length of the miniature device 100 is increased from L1 (shown in FIG. 6A) to L2 (shown in FIG. 6B). This may facilitate a cutting operation, in particular when the abutting end 104 bears against tissue opposite the treatment site, for example as described below.

As illustrated in FIGS. 7A and 7B, the miniature device 100 may be provided such that the cutting portion 106 is disposed in the center of the longitudinal axis X, the hydrogel element 124 being configured to push it radially, thereby exposing the cutting portion to perform a cutting operation.

According to some examples, the miniature device 100 is designed for a therapeutic activity which includes movement along a path within the SAS or SDS. It may be formed having a height which is approximately one to two times the distance, and/or a length which approximately two to four times the distance, between the arachnoid mater and the pia mater (in the case of a path which includes the SAS) or between the dura mater and the arachnoid mater (in the case of a path which includes the SDS).

The system 10 may be used to perform a therapeutic activity by the miniature device 100 at a treatment site close to (e.g., within several millimeters of) the surface of the parenchyma, such as at treatment site which is typically accessed using burr holes formed in the skull of the patient. According to some examples, the treatment site is in the supratentorial region, for example above the frontal lobe. According to some examples, the therapeutic activity is performed to administer a treatment for a glioblastoma multiform. The therapeutic activity may further comprise removal of the miniature device 100 from the patient.

The miniature device 100 of the system 10 is introduced into the SAS of the patient, for example via a needle. This may be done at any suitable location, e.g., at the cisterna magnum or along the spine.

Subsequently, the driving device 200 operates to produce a varying magnetic field configured to direct the miniature device 100 along a suitable route within the SAS to the treatment site. According to some examples, this includes operating the driving device 200 to move the miniature device 100 within the SAS around one side of the temporal lobe, and continue to a treatment site adjacent the frontal lobe, in the SAS between the skull and the parenchyma. According to other examples, this includes operating the driving device 200 to move the miniature device 100 within the SAS along the dorsal side of the cerebellum, past the occipital and parietal lobes, to a treatment site adjacent the frontal lobe, in the SAS between the skull and the parenchyma. It will be appreciated that while specific routes are described herein to reach a treatment site adjacent the frontal lobe, other routes may be traversed and/or other treatment sites accessed, without departing from the scope of the presently disclosed subject matter, mutatis mutandis.

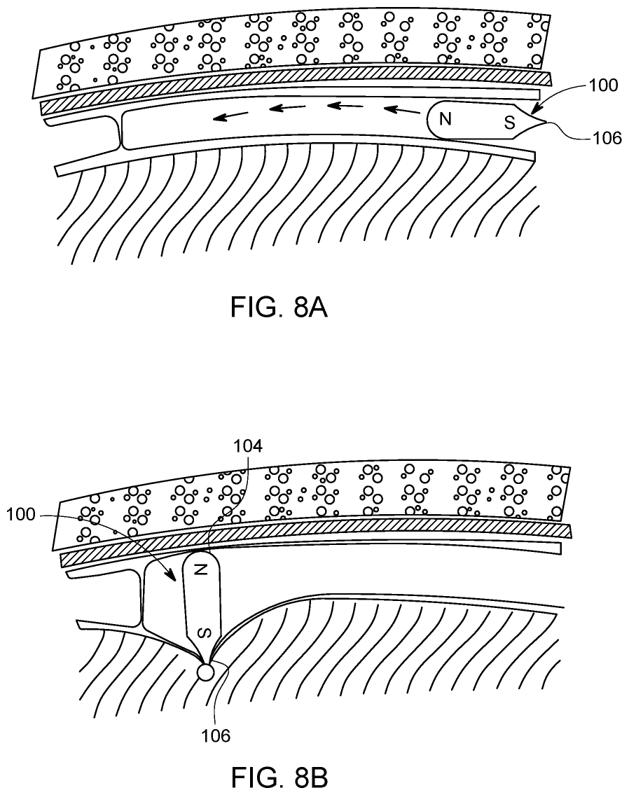

As illustrated in FIG. 8A, according to some examples, the driving device 200 operates to move the miniature device 100 such that its longitudinal axis X is substantially aligned with the direction of travel at its location. According to some examples, the driving device 200 operates to move the miniature device 100 such that its abutting end 104 constitutes the leading edge, i.e., such that the miniature device moves abutting end-first.

As illustrated in FIG. 8B, once the miniature device 100 has reached the treatment site, the driving device 200 operates to rotate it in place, such that its cutting portion 106 contacts the pia mater. As the length of the body of the miniature device 100 is greater than the distance between the skull of the patient and the pia mater, the area of the skull opposite the treatment site serves as a contra, i.e., the abutting end 104 of the miniature device bears against it, creating pressure where the cutting portion 106 contacts the pia mater and facilitating a cutting operation, e.g., puncturing, of the pia mater at the point of contact.

According to some examples, the driving device 200 may be configured to operate to wiggle the miniature device 100 and/or to rotate the miniature device about its longitudinal axis X in order to facilitate the cutting operation.

According to some examples, the miniature device 100 carries a therapeutic component, for example as is known in the art, and delivers it to the treatment site, e.g., after the cutting operation has taken place. Accordingly, the miniature device 100 is able to move within the meninges surrounding the parenchyma, and breach the pia at and/or adjacent a treatment site to deliver a therapeutic component thereto. This may be useful, e.g., to eliminate the need for forming a burr hole to access the treatment site.

It will be recognized that examples, embodiments, modifications, options, etc., described herein are to be construed as inclusive and non-limiting, i.e., two or more examples, etc., described separately herein are not to be construed as being mutually exclusive of one another or in any other way limiting, unless such is explicitly stated and/or is otherwise clear. Those skilled in the art to which this invention pertains will readily appreciate that numerous changes, variations, and modifications can be made without departing from the scope of the presently disclosed subject matter, mutatis mutandis.

1. A system for facilitating performing a therapeutic activity at a predetermined treatment site in a patient, the system comprising:
an elongated miniature device extending along a longitudinal axis spanning between a distal cutting end formed with a sharp cutting portion, and a blunt proximal abutting end, the elongated body comprising a substantially non-convex bottom surface extending substantially between the cutting end and the abutting end; and
a driving device configured to generate a varying magnetic field to remotely control motion of the miniature device.

2. The system according to claim 1, the miniature device defining first and second lateral planes being perpendicular to each other and mutually perpendicular to the longitudinal axis, wherein the widest width of the miniature device along the direction of the first lateral plane is at least twice the largest height of the miniature device along the direction of the second lateral plane.

3. The system according to claim 2, wherein for each point along a majority of the longitudinal axis, the width of the miniature device along the direction of the first lateral plane is at least twice the height of the miniature device along the direction of the second lateral plane.

4. The system according to claim 1, wherein the bottom surface of the miniature device is concave.

5. The system according to claim 1, wherein the bottom surface of the miniature device is substantially planar.

6. The system according to claim 1, wherein, in a direction perpendicular to the longitudinal axis, the width of the bottom surface spans a majority of the width of the miniature device, along a majority of the length of the miniature device.

7. The system according to claim 6, wherein the width of the bottom surface is the width of the miniature device, along a majority of the length of the miniature device.

8. The system according to claim 1, wherein the cutting portion of the miniature device is covered by a coating configured to dissolve within the patient after a predetermined amount of time has passed, thereby exposing the cutting portion.

9. The system according to claim 1, the cutting portion of the miniature device comprising one or more barbs configured to inhibit removal of cutting portion from a tissue pierced thereby.

10. The system according to claim 1, the miniature device comprising a shoulder formed proximally to and facing the cutting portion.

11. The system according to claim 1, the miniature device comprising a distal cutting element comprising the cutting end, a proximal abutting element comprising the abutting end, and a linking element connecting the cutting and abutting elements, the linking element being configured to selectively disconnect the cutting and abutting elements.

12. The system according to claim 11, wherein the linking element comprises a material configured to dissolve within the patient after a predetermined amount of time has passed, thereby disconnecting the cutting and abutting elements.

13. The system according to claim 1, the miniature device comprising an external thread formed at least on a distal end thereof.

14. The system according to claim 1, the miniature device comprising a hydrogel configured, when exposed to water, to extend the miniature device along the longitudinal axis.

15. The system according to claim 1, wherein the miniature device is covered with a lubricious coating.

16. The system according to claim 1, wherein the miniature device comprises a magnetic material.

17. The device according to claim 16, the magnetic material being magnetized along the longitudinal axis.

18. The system according to claim 1, wherein the driving device is designed to generate the varying magnetic to move the miniature device to a predefined location in the patient, and to rotate it at the location.

19. The system according to claim 18, wherein the driving device is designed to generate the varying magnetic to rotate the miniature device such that the cutting portion thereof contacts a treatment site of a tissue in the patient, and the abutting end bears upon a contra location of a tissue opposite the treatment site, thereby facilitating piercing at the treatment site by the cutting portion.

20. The system according to claim 19, wherein the tissue opposite the treatment site is a hard tissue.

21. The system according to claim 1, wherein the driving device is designed to generate the varying magnetic to move the miniature device within the patient such that it is oriented substantially along its longitudinal axis, wherein the abutting end of the miniature device constitutes a leading edge thereof, at least while moving toward the treatment site.

22. The system according to claim 1, wherein the therapeutic activity comprises delivery of one or more therapeutic components.

23. The system according to claim 1, wherein the therapeutic activity comprises performing one or more cutting operations.

24. The system according to claim 1, wherein the therapeutic activity comprises treatment of a glioblastoma multiform.
